(19) United States
(12) Patent Application Publication
(10) Pub. No.: US 2006/0288449 A1
(43) Pub. Date: Dec. 21, 2006

(54) Process for purifying target compounds from plant sources using ceramic filtration

(76) Inventors: Stephen J. Garger, Vacaville, CA (US); Barry Bratcher, Owensboro, KY (US); Fakhrieh S. Vojdani, Davis, CA (US)

Correspondence Address: LARGE SCALE BIOLOGY CORPORATION, 3333 VACA VALLEY PARKWAY, SUITE 1000, VACAVILLE, CA, 95688, US

(21) Appl. No.: 11/301,469

(22) Filed: Dec. 12, 2005

Related U.S. Application Data
(63) Continuation-in-part of application No. 11/249,685, filed on Oct. 12, 2005.
(60) Provisional application No. 60/618,485, filed on Oct. 12, 2004.
(60) Provisional application No. 60/635,214, filed on Dec. 10, 2004.

(51) Int. Cl. A01H 1/00; C12N 7/02; C12N 15/82; C07K 14/415

(52) U.S. Cl. 800/280; 435/239; 800/317.3; 530/370

(57) ABSTRACT

The present invention describes a method for isolating a target compound from a plant, the method comprising obtaining a plant extract, passing such plant extract through a ceramic filter to obtain a permeate, and purifying the target compound from such permeate. This method, among other things, allows ultrafiltration of crude plant extracts, such as green juice homogenates.

### CROSS REFERENCE TO RELATED APPLICATIONS

This application claims the benefit of provisional Application No. 60/635,214, filed on Dec. 10, 2004. This application is also a continuation in part of application Ser. No. 11/249,685, filed on Oct. 12, 2005, which claims the benefit of provisional Application No. 60/618,485, filed on Oct. 12, 2004. The above-referenced applications are incorporated herein by reference.

### BACKGROUND OF THE INVENTION

Various publications and patents are referred to throughout this application. Each of these publications or patents is incorporated by reference herein.

The present invention relates to a process for isolating and purifying target compounds, such as proteins, peptides and viruses, from plants. More specifically, the present invention is capable of being scaled up to commercial levels.

Plant proteins and enzymes have long been exploited for many purposes, from viable food sources to biocatalytic reagents, or therapeutic agents. During the past decades, the development of transgenic and transfected plants and improvements in genetic analysis have brought renewed scientific significance and economical incentives to these applications. The concepts of molecular plant breeding and molecular plant farming, wherein a plant system is used as a bioreactor to produce recombinant bioactive materials, have received great attention.

Many examples in the literature have demonstrated the utilization of plants or cultured plant cells to produce active mammalian proteins, enzymes, vaccines, antibodies, peptides, and other bioactive species. Ma et al. (Science 268: 716-719 (1995)) were the first to described the production of a functional secretory immunoglobulin in transgenic tobacco. Genes encoding the heavy and light chains of murine antibody, a murine joining chain, and a rabbit secretory component were introduced into separate transgenic plants. Through cross-pollination, plants were obtained to co-express all components and produce a functionally active secretory antibody. In another study, a method for producing antiviral vaccines by expressing a viral protein in transgenic plants was described (Mason et al., Proc. Natl. Acad. Sci. U.S.A. 93: 5335-5340 (1996)). The capsid protein of Norwalk virus, a virus causing epidemic acute gastroenteritis in humans was shown to self-assemble into virus-like particles when expressed in transgenic tobacco and potato. Both purified virus-like particles and transgenic potato tubers when fed to mice stimulated the production of antibodies against the Norwalk virus capsid protein.

Alternatively, the production and purification of a vaccine may be facilitated by engineering a plant virus that carries a mammalian pathogen epitope. By using a plant virus, the accidental shedding of virulent virus with the vaccine is abolished, and the same plant virus may be used to vaccinate several hosts. For example, malarial epitopes have been presented on the surface of recombinant tobacco mosiac virus (TMV) (Turpen et al., BioTechnology 13:53-57 (1995)). Selected B-cell epitopes were either inserted into the surface loop region of the TMV coat protein or fused into the C-terminus. Tobacco plants after infection contain high titers of the recombinant virus, which may be developed as vaccine subunits and readily scaled up. In another study aimed at improving the nutritional status of pasture legumes, a sulfur-rich seed albumin from sunflower was expressed in the leaves of transgenic subterranean clover (Khan, et al., Transgenic Res. 5:178-185 (1996)). By targeting the recombinant protein to the endoplasmic reticulum of the transgenic plant leaf cells, an accumulation of transgenic sunflower seed albumin up to 1.3% of the total extractable protein could be achieved.

Work has also been conducted in the area of developing suitable vectors for expressing foreign genetic material in plant hosts. Ahlquist, U.S. Pat. No. 4,885,248 and U.S. Pat. No. 5,173,410 described preliminary work done in devising transfer vectors which might be useful in transferring foreign genetic material into plant host cells for the purpose of expression therein. Additional aspects of hybrid RNA viruses and RNA transformation vectors are described by Ahlquist et al. in U.S. Pat. Nos. 5,466,788, 5,602,242, 5,627,060 and 5,500,360. Donson et al., U.S. Pat. No. 5,316,931 and U.S. Pat. No. 5,589,367, demonstrate for the first time plant viral vectors suitable for the systemic expression of foreign genetic material in plants. Donson et al. describe plant viral vectors having heterologous subgenomic promoters for the systemic expression of foreign genes. The availability of such recombinant plant viral vectors makes it feasible to produce proteins and peptides of interest recombinantly in plant hosts.

Elaborate methods of plant genetics are being developed at a rapid rate and hold the promise of allowing the transformation of virtually every plant species and the expression of a large variety of genes. However, in order for plant-based molecular breeding and farming to gain widespread acceptance in commercial areas, it is necessary to develop a cost-effective and large-scale purification system for the bioactive species produced in the plants, either proteins or peptides, especially recombinant proteins or peptides, or virus particles, especially genetically engineered viruses.

Some processes for isolating proteins, peptides and viruses from plants have been described in the literature (Johal, U.S. Pat. No. 4,400,471, Johal, U.S. Pat. No. 4,334,024, Wildman et al., U.S. Pat. No. 4,268,632, Wildman et al., U.S. Pat. No. 4,289,147, Wildman et al., U.S. Pat. No. 4,347,324, Hollo et al., U.S. Pat. No. 3,637,396, Koch, U.S. Pat. 4,233,210, and Koch, U.S. Pat. No. 4,250,197, the disclosure of which are herein incorporated by reference). The succulent leaves of plants, such as tobacco, spinach, soybean, and alfalfa, are typically. composed of 10-20% solids, the remaining fraction being water. The solid portion is composed of a water soluble and a water insoluble portion, the latter being predominantly composed of the fibrous structural material of the leaf. The water soluble portion includes compounds of relatively low molecular weight (MW), such as sugars, vitamins, alkaloids, flavors, amino acids, and other compounds of relatively high MW, such as natural and recombinant proteins.

Proteins in the soluble portion of the plant bombast can be further divided into two fractions. One fraction comprises predominantly a photosynthetic protein, ribulose 1,5-diphosphate carboxylase (or RuBisCO), plant organelles, such as chloroplasts, cell membrane and other cell debris. The molecular weight of RuBisCO subunit is 550 kDa. The RuBisCO large subunit has a molecular weight of 55 kDa, and the small subunit has a molecular weight of 14 kDa. The whole complex contains eight of each subunit. This fraction is commonly referred to as “Fraction 1.” RuBisCO is abundant, comprising up to 25% of the total protein content of a leaf and up to 10% of the solid matter of a leaf. The other fraction contains a mixture of proteins and peptides whose subunit molecular weights typically range from about 3 kD to 100 kD and other compounds including sugars, vitamins, alkaloids, flavors and amino acids. This fraction is collectively referred to as “Fraction 2.” Proteins in Fraction 2 can be native host materials or recombinant materials including proteins and peptides produced via transfection or transgenic transformation. Transfected plants may also contain virus particles having a molecular size greater than 1,000 kD.

One process for isolating target compounds from plants begins with disintegrating leaf bombast and pressing the resulting pulp to produce “green juice.” The process is typically performed in the presence of a reducing agent or antioxidant to suppress unwanted oxidation. The green juice contains various protein components and fine particulate green pigmented material. The green juice may be pH adjusted and heat treated. One method subjects the pH adjusted, heat-treated green juice to a centrifugation step that separates the Fraction 1 and Fraction 2 components. See, for example, U.S. Pat. No. 6,037,456. This method will be referred to herein as the centrifugation method, and supernatant obtained from such centrifugation step will be referred to as S1. This centrifugation step may be scaled up, but for certain product purifications better results are obtained with the purification method of the present invention. This is because equipment limitations, based upon the required G x time to affect feedstream clarification can result in impractical processing times or reductions in the volume of extract that can be processed at once. In addition, the methods of the present invention may be more effective than centrifugation in removing recombinant virions if a transient-based system is used for target compound expression.

Filtration steps are also commonly used in order to purify target compounds from plants, typically in “downstream” purification processes, i.e., after Fraction 1 components have been removed from the green juice. It has not been practical to use filters in an initial step to purify green juice because the Fraction 1 components quickly foul the typical membranes used for ultrafiltration: cellulose, cellulose acetate and membranes composed of polymers, such as polyether sulfone, polyvinylidene fluoride and polyamide. Membranes, such as those manufactured from polyether sulfone, may also develop a charge that will interfere with size filtration. Even when ultrafiltration is used in downstream steps or with cleaner plant extracts, such as the interstitial fluid extracts described in U.S. Pat. No. 6,284,875, non-ceramic membranes may foul easily, build up a charge, and cannot withstand harsh pH conditions, leading to lower target compound recovery. Currently available membranes are also easily damaged during harsh cleaning processes.

### SUMMARY OF THE INVENTION

The present invention addresses these problems through a method involving ceramic filtration. Ceramic membranes are strong, inert (such that they do not build up a charge during filtration), and are resistant to fouling and damage during use and, especially, during cleaning. Use of ceramic filters allows introduction of an ultrafiltration step at an early stage of purification of a target compound from plant materials. For example, the ceramic filter may be used in an “upstream” step to remove Fraction 1 components and other large particles, such as virus, from a crude plant extract. In one embodiment, green juice is passed through the ceramic membrane, resulting in a permeate that is of sufficient clarity and purity to be concentrated via ultrafiltration after just one step. In contrast, supernatants from centrifugation of green juice typically require additional clean-up steps in order to minimize membrane fouling during ultrafiltration.

One article discusses using ceramic filters with very small pore size (MWCO of 1 kDa to 50 kDa) to obtain and concentrate plant protein from green juice but does not apply this method to purifying specific target compounds from the retentate or the permeate. Instead, the researchers' goal was concentration of all or substantially all plant protein. See Koschuh, W., et al., Desalination 163: 253-259 (2004).

In other embodiments, the ceramic filter is used in place of other types of filters to avoid fouling problems and, surprisingly, to achieve high recovery rates with larger pore sizes than used with previous membranes in ultrafiltration steps.

The present invention features a method for isolating a target compound from a plant by obtaining a plant extract, passing such plant extract through a ceramic filter and purifying the target compound from a permeate created by such filtration. The plant extract may be, for example, an interstitial fluid extract or a crude plant extract, such as a green juice homogenate.

Ceramic filters may have a pore size of equal to or less than 5 microns, equal to or less than 1 micron, equal to or less than 0.2 micron or equal to or less than 0.1 micron. Alternatively, the plant extract may be passed through more than one ceramic filter arranged in a series. In one embodiment, the ceramic filters arranged in series have pore sizes of 0.1 micron and 0.2 micron.

In some embodiments the step of passing the plant extract through a ceramic filter may also include washing one or more times a concentrate created by the ceramic filtration.

This invention encompasses plants in which the target compound is expressed by a transgene and plants infected with a viral vector that encodes the target compound. In a preferred embodiment, the viral vector is tobacco mosaic virus.

In some embodiments the target compound is a protein, such as aprotinin, or an. antiviral protein, such as griffithsin. In a preferred embodiment, the target compound is a soluble protein. In other embodiments, it is a sugar, vitamin, alkaloid, flavor or amino acid.

In instances in which the plant extract is a green juice homogenate, a target compound is isolated from a plant by homogenizing plant tissue to produce a green juice homogenate, passing such green juice homogenate through a ceramic filter, and purifying the target compound from a permeate created from such filtration.

In one aspect of this invention, the pH and/or ionic content of the green juice homogenate may be adjusted such that the target compound is soluble. Such adjustment may occur before or after homogenization. Adjustments of the homogenate to (i) neutral to acidic pH, (ii) pH of equal to or less than about 7, (iii) pH of equal to or less than about 6.0, or (iv) pH of equal to or less than about 5.2 are contemplated by this invention.

In another aspect the green juice homogenate is temperature adjusted. In some embodiments, temperature adjustment is in addition to the adjustments described above to attain a soluble target compound. Temperature may be adjusted to greater than about 40° C., between about 45° C. and 65° C. and between about 45° C. and 50° C.

The above-described step of purifying the target compound from a permeate obtained through ceramic filtration of a plant extract may be accomplished by any purification method known in the art, including one or more of the following: ultrafiltration, chromatography, an affinity-based method of purification, salt precipitation, or polyethylene glycol precipitation or crystallization. In one embodiment, the purification step comprises subjecting the permeate to ultrafiltration, typically through a low molecule weight cut off membrane chosen in light of the target compound. In another embodiment the purification step comprises subjecting the permeate to cation exchange chromatography, preferably an SP Sepharose column. In another embodiment, the-permeate is subjected to reversed phase chromatography, preferably a RPC 15 or RPC 30 column.

The present invention also contemplates a method for purifying large molecular weight molecules, such as virus, by passing a plant extract through a ceramic filter of appropriate pore size, such as less than or equal to 0.5 micron, 0.2 micron, or 0.1 micron. The large molecular weight molecule is retained by the membrane. One embodiment involves retained virus, which may be further purified by salt precipitation, polyethylene glycol precipitation or crystallization. In a preferred embodiment, the virus is tobacco mosaic virus.

These and other features and advantages of this invention are described in, or are apparent from, the following detailed description of various exemplary embodiments of the devices and methods according to this invention.

### BRIEF DESCRIPTION OF THE DRAWINGS

Various exemplary embodiments of this invention will be described in detail, with reference to the following figures, wherein:

FIG. 1 illustrates ceramic filtration of rAprotinin at pH 4 at pilot scale. Using ceramic filtration technique removes very efficiently TMV, RuBisCO, as well as other large proteins. Lane 2: Sample from green juice containing rAprotinin. Lane 3: Green juice after centrifugation at pH 4 to remove RuBisCO. Lane 4: Ceramic permeate. Samples were separated by SDS-PAGE using a 16%Tris Glycine gel and stained with Coomassie Brilliant Blue stain. Lane 1 shows molecular marker (Mark 12, Invitrogen) with corresponding molecular weigh set forth at left. The gel mobility of TMV coat protein, rAprotinin, large and small subunits of RuBisCO are highlighted at the right.

FIG. 2 illustrates ceramic clarification of rGriffithsin (rGRFT) at pH 6 at pilot scale. Lane 2: Sample from green juice containing rGRFT after centrifugation at pH6 to remove RuBisCO. Lane 3: Ceramic permeate. Samples were separated by SDS-PAGE using a 10-20% Tris Glycine gel and stained with Coomassie Brilliant Blue stain. Lane 1 shows molecular marker (Mark 12, Invitrogen) with corresponding molecular weigh shown at left. The TMV coat protein and rGRFT protein are highlighted at the right. Coat protein and RuBisCO are completely removed by ceramic filtration technique from the green juice.

FIG. 3 illustrates ceramic clarification of rAprotinin at pH 4 at pilot scale using 0.1 and 0.2 micron ceramic membranes. Green juice containing rAprotinin was made from field grown N. excelsiana tissue and passed through a ceramic filters of 0.1 and 0.2 micron pore size for clarification. Plant cell particulate, RuBisCO, TMV, as well as other large and insoluble proteins were removed efficiently from the green juice. Lane 1: Sample of 0.2 micron ceramic permeate. Lane 2: Sample of 0.1 micron ceramic permeate. Lane 3: Green juice clarified by centrifugation (S1). Samples were separated by SDS-PAGE using a 10-20% Tris Glycine gel and stained with Coomassie Brilliant Blue stain. Lane 4 shows molecular marker (Mark 12, Invitrogen) with corresponding molecular weighs shown at left. The gel mobility of TMV coat protein, rAprotinin, large and small subunits of RuBisCO are highlighted at left.

### DETAILED DESCRIPTION OF THE INVENTION

Ceramic membranes are used in the present invention to purify and concentrate target compounds from plant materials. Generally, the method comprises purifying a target compound, such as a protein, peptide, or virus, from a plant extract by passing the plant extract through a ceramic filter and purifying the target compound from the permeate. As referred to herein, a plant extract refers to any material derived from a plant or a part thereof, such as leaves, seeds, and tubers. Examples of plant extracts are green juice homogenate and interstitial fluid, described in more detail below, or the supernatant or pellet obtained by the heat treatment, pH adjustment and centrifugation steps described in U.S. Pat. No. 6,037,456. As described in the Examples below, the ceramic filtration method may be applied on a small scale and is easily scaled up from bench-scale to pilot-scale or large-scale. Pilot-scale typically involves between about 10 kg to 1000 kg plant material, and large-scale typically involves equal to or greater than about 1000 kg plant material, preferably equal to or greater than about 3000 kg.

Generally, the method comprises passing the plant extract, such as green juice homogenate or interstitial fluid, through a ceramic filter having one or more membranes each with a pore size of about 5 microns or less. In one embodiment, the pore size is less than or equal to about 1 micron. In a preferred embodiment, the pore size is less than or equal to about 0.2 micron. A pore size of less than or equal to about 0.1 micron is particularly preferred. Any ultrafiltration with a pore size of equal to or less than 0.2 micron also serves as a bioburden reduction step, as microbes are retained at this pore size. See U.S. Pat. No. 5,242,595.

The plant extract may be applied to the membrane in cross-flow filtration in order to allow processing of more material through fewer square feet of membrane. The high flow of feed continually cleans the membrane. In some embodiments, such as small scale purifications, dead-end filtration may also be used effectively.

Any ceramic membrane of appropriate pore size may be used in this invention. Such membranes are available from, e.g., Pall Life Sciences (East Hills, N.Y.) and TAMI Industries (Nyons, France). In a preferred embodiment, the ceramic membrane is that described in the Examples section below. A ceramic filter (or module), as that term is used herein, includes one or more ceramic membranes of the same pore size, as described in detail in the Examples section below. In some embodiments, more than one ceramic filter will be arranged in series, such that the permeate passes through more than one ceramic membranes of the same pore size, of diminishing pore size or of increasing pore size.

The filtration system may also comprise a means for cleaning the membrane periodically during runs, such as via a back-pulsing device that briefly back pressures the membrane to dislodge any accumulated gel layer or solids.

The concentrate resulting from the filtration step may be washed and the resulting permeate collected. The term washed, as used herein, refers to washing the concentrate with liquid, such as the extraction solution or a slight variation of the extraction solution, e.g., with added salt, and then passing such liquid back though the ceramic filter to obtain more permeate. Preferably, a plurality of washes are performed to optimize recovery of target compound in the permeate. In one embodiment, the percentage of target compound recovered in the permeate, with or without washes, is at least about 65%, preferably at least about 75%, more preferably at least about 80%.

In one embodiment, the plant extract from which target compound is isolated is a crude plant extract. A crude plant extract, as used herein, is an extract in which plant cells have been initially disrupted without further purification, such as a green juice homogenate. Green juice homogenate is obtained by homogenizing the subject plant material in an extraction solution. Plant leaves may be disintegrated using any appropriate machinery or process available. For instance, a Waring blender for a small scale purification or a Reitz disintegrator for a large scale purification has been successfully used in some embodiments of the instant invention. The homogenized mixture may then be pressed using any appropriate machinery or process available. For example, a screw press for a large scale or a cheesecloth for a small scale has been successfully employed in some embodiments of the instant invention. The extraction solution may be a buffer adjusted to a certain pH. The extraction solution may also include one or more of the following components: salt to adjust its ionic strength, a suitable reducing agent or antioxidant to suppress unwanted oxidation, and detergent. Exemplary extraction solutions are described in the Examples. Sodium metabisulfite is successfully used in some embodiments of the instant invention as a reducing agent and antioxidant. The product obtained from this procedure shall be referred to herein as green juice or green juice homogenate.

In some embodiments, the pH or ionic concentration of the green juice homogenate is adjusted to attain conditions in which the target compound is soluble. This adjustment may take place before or after homogenization. If before, the adjustment will be accomplished via the extraction solution. Detergents may also be used to assist in solubilizing the target compound. Attaining the proper conditions to obtain a soluble protein is a matter of routine experimentation for one of skill in the art.

In some instances, the pH of the green juice homogenate is adjusted so that the homogenate is neutral or slightly acidic, preferably to about pH 7.5 or less. In another embodiment, the extract is adjusted to an acidic pH, preferably at or below 6.5, more preferably, at or below about 5.2. At a pH less than 5.2, RuBisCO tends to coagulate, which assists in its retention by the membrane. In the green juice centrifugation method mentioned above, pH 5.2 is preferred so that the RuBisCO falls out of solution. Because, however, the ceramic membrane separates by size rather than by solubility, pH of 5.2 is not necessary to obtain effective separation, as, illustrated in Example 3, below, and may not be feasible given the nature of the target protein.

If the target protein is stable at higher temperatures, the green juice homogenate may be heat treated after the solution is adjusted to attain conditions in which the target compound is soluble. In one embodiment, the green juice homogenate is heated to at least about 45° C., preferably between about 45° C. to 65° C., more preferably to about 45° C. to 50° C.

In another embodiment, the plant extract is an interstitial fluid extract. Such extract may be obtained as described in U.S. Pat. No. 6,284,875, by infiltrating plant foliage with a buffer solution by subjecting the submerged plant foliage to a substantially vacuum environment, removing the excess liquid from the plant foliage after exposing the foliage to the substantially vacuum environment, and centrifuging the foliage. As a result of such procedure, large amounts of desirable proteins may be removed from the interstitial space of plants thereby making it feasible to isolate both naturally-occurring and recombinantly produced proteins from plant foliage in commercial-scale quantities without homogenizing the plant cells. The fluid resulting from centrifuging the foliage shall be referred to herein as interstitial fluid. The interstitial fluid may be pH and temperature adjusted, as described above for the green juice homogenate.

Other plant extracts include S1 obtained from the centrifugation method, as defined above in the Background of Invention section, and plant extracts that have been partially purified by methods other than the centrifugation method, after initial cellular disruption.

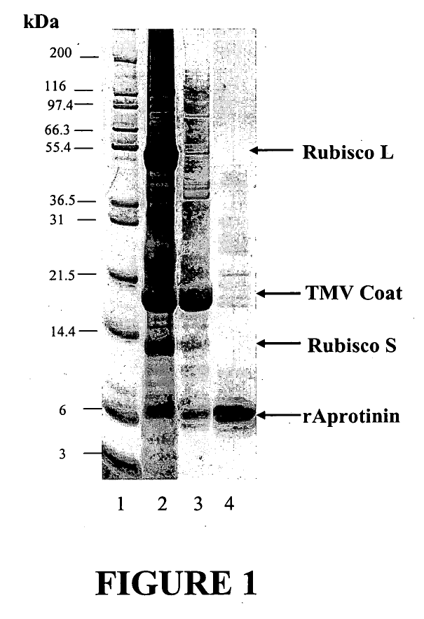

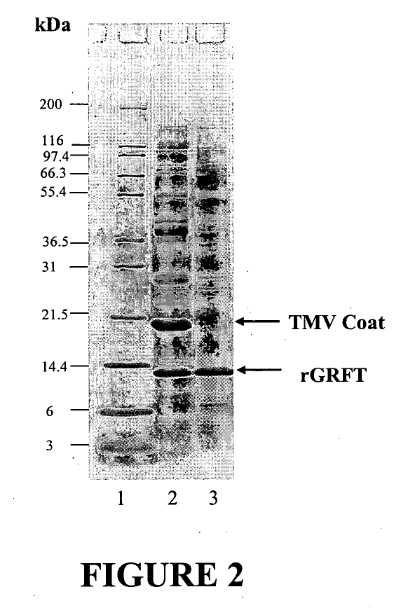

In one embodiment the target compound is a protein of less than about 200 kDa, preferably less than or equal to about 150 kDa and more preferably less than or equal to about 150 kDa. As shown in FIGS. 1 and 2, small molecular weight compounds, such as Fraction 2 compounds, typically flow through the membrane while larger components, such as Fraction 1 components and virus, if present, are typically retained by the membrane, resulting in substantial purification of the target compound, preferably such that the permeate comprises at least about 65% pure target compound, preferably at least about 70% pure target compound and more preferably at least about 75% pure target compound.

The invention is also specifically intended to encompass embodiments wherein the peptide or protein of interest is selected from the group consisting of IL-1, IL-2, IL-3, IL-4, IL-5, IL-6, -IL-7, IL-8, IL-9, IL-10, IL-11, IL-12, EPO, G-CSF, GM-CSF, hPG-CSF, M-CSF, Factor VIII, Factor IX, tPA, receptors, receptor antagonists, antibodies, single-chain antibodies, enzymes, neuropolypeptides, insulin, antigens, vaccines, aprotinin, peptide hormones, calcitonin, antiviral proteins, such as griffithsin, and human growth hormone. In yet other embodiments, the protein or peptide of interest may be an antimicrobial peptide or protein consisting of protegrins, magainins, cecropins, melittins, indolicidins, defensins, beta.-defensins, cryptdins, clavainins, plant defensins, nicin and bacterecins. These and other proteins and peptides of interest may be naturally produced or produced by recombinant methodologies in a plant.

The target compounds may also be sugars, vitamins, alkaloids, flavors, amino acids, which are small molecular weight compounds that will be present in the permeate.

Once in the permeate, the target compound may be concentrated and purified according to any suitable purification procedures. For example, the target compound may be further purified by a series of low molecular weight cutoff ultrafiltration and other methods, which are well known in the art. Ultrafiltration is typically performed using a MWCO membrane in the range of about 1 to 500 kD according to methods well known in the art. In some embodiments of the instant invention, a large MWCO membrane is first used to filter out the residual virus and other host materials, although depending on the pore size of the ceramic membrane, this may not be necessary as nearly all Fraction 1 protein and/or virus may be removed in the ultrafiltration step. Large molecular weight components may remain in the concentrates. Filtrates containing the proteins/peptides of interest may be optionally passed through another ultrafiltration membrane, typically of a smaller MWCO, such that the target compound can be collected in the concentrates. Additionally cycles of ultrafiltration may be conducted, if necessary, to improve the purity of the target compound. The choice of MWCO size and ultrafiltration conditions depends on the size of the target compound and is an obvious variation to those skilled in the art. The ultrafiltration step generally results in a reduction in process volume of about 10- to 30-fold or more and allows diafiltration to further remove undesired molecular species.

Other procedures that may be used in addition to or in lieu of ultrafiltration may include but are not limited to protein precipitation, salt precipitation, polyethylene glycol precipitation, crystallization, expanded bed chromatography, anion exchange chromatography, cation exchange chromatography, hydrophobic-interaction chromatography, HPLC, FPLC and affinity chromatography. A general discussion of some protein purification techniques is provided by Jervis et al., Journal of Biotechnology 11:161-198 (1989).

In one embodiment of the present invention, the permeate from ceramic filtration is subject to cation exchange chromatography, preferably using a SP Sepharose column. The eluant from such chromatography may then be subjected to further chromatography procedures, such as reverse phase chromatography, preferably using a 15 μm RPC or 30 μm RPC column.

This ceramic filtration method may also be used to purify or concentrate virus or other large molecular weight compounds from plant extracts. In one embodiment, a plant extract is passed through the ceramic membrane and virus or large molecular weight compounds are retained. In a preferred embodiment, the plant extract is at least partially purified before it is applied to the ceramic membrane. In a particularly preferred embodiment, the plant extract is S1.

Large molecular weight molecules, which are typically greater than 400 kDa, including virus, may be further purified from this retentate using any of the purification methods described above, including PEG or salt precipitation or crystallization, although this may also serve as the final step in purification.

The virus of interest may be a potyvirus, a tobamovirus, a bromovirus, a armovirus, a luteovirus, a marafivirus, the MCDV group, a necrovirus, the PYFV group, a sobemovirus, a tombusvirus, a tymovirus, a capillovirus, a closterovirus, a carlavirus, a potexvirus, a comovirus, a dianthovirus, a fabavirus, a repovirus, a PEMV, a furovirus, a tobravirus, an AMV, a tenuivirus, a rice necrosis virus, caulimovirus, a geminivirus, a reovirus, the commelina yellow mottle virus group and a cryptovirus, a Rhabovirus, or a Bunyavirus.

In a preferred embodiment, the virus is tobacco mosaic virus. As described in Example 4, below, tobacco mosaic virus was substantially retained when passed through a 0.1 micron membrane. This was surprising, as previous ultrafiltration of virus-containing plant extract has been accomplished using cellulose membranes with much smaller pore sizes.

While this invention has been described in conjunction with the specific embodiments outlined above, it is evident that many alternatives, modifications and variations will be apparent to those skilled in the art. Accordingly, the preferred embodiments of the invention, as set forth above, are intended to be illustrative, not limiting. Various changes may be made without departing from the spirit and scope of this invention.

Definitions

In order to provide an even clearer and more consistent understanding of the specification and the claims, including the scope given herein to such terms, the following definitions are provided:

A “virus” is defined herein to include the group consisting of a virion wherein said virion comprises an infectious nucleic acid sequence in combination with one or more viral structural proteins; a non-infectious virion wherein said non-infectious virion comprises a non-infectious nucleic acid in combination with one or more viral structural proteins; and aggregates of viral structural proteins wherein there is no nucleic acid sequence present or in combination with said aggregate and wherein said aggregate may include virus-like particles (VLPs). Said viruses may be either naturally occurring or derived from recombinant nucleic acid techniques and include any viral-derived nucleic acids that can be adopted whether by design or selection, for replication in whole plant, plant tissues or plant cells.

A “virus population” is defined herein to include one or more viruses as defined above wherein said virus population consists of a homogeneous selection of viruses or wherein said virus population consists of a heterogenous selection comprising any combination and proportion of said viruses.

“Virus-like particles” (VLPs) are defined herein as self-assembling structural proteins wherein said structural proteins are encoded by one or more nucleic acid sequences wherein said nucleic acid sequence(s) is inserted into the genome of a host viral vector.

“Protein and peptides” are defined as being either naturally-occurring proteins and peptides or recombinant proteins and peptides produced via transfection or transgenic transformation.

### EXAMPLES Example 1: Purification of Aprotinin from Plant Materials—Process Development

Nicotiana excelsiana plants were inoculated with TMV-based recombinant aprotinin (rAprotinin) virion construct 2602. Plasmid pLSB2602 contains the mature bovine Aprotinin-coding region. Its cloning is described in detail in U.S. patent application Ser. No. 11/249,685. In addition, it has been deposited in accordance with the terms of the Budapest Treaty, as described at the end of this section.

Inoculum solution was delivered to the plants via an air-assisted inoculation process. Each plant was sprayed with the inoculum solution at approximately 75 psi air pressure. The inoculum solution contains the rAprotinin virion construct, a Na(KPO₄) buffer, diatomaceous earth, and purified water.

Plants were harvested by cutting at the base of the plant using hand pruners. Harvested plant biomass (100-500 kg tissue) was disintegrated by passing through a Corenco (model M8A-D, Sebastopol, Calif.). Buffer solution (consisting of sodium chloride, ascorbic acid, sodium metabisulfite) was added to plant biomass at a ratio of 0.5 L of buffer per kg of plant biomass. Plant biomass was then passed through the disintegrator a second time. Homogenized plant biomass was then processed through a Vincent horizontal screw press (model VP-1, Tampa, Fla.) to extract the liquid from the homogenized plant biomass. Extracted liquid was pumped into a water-jacketed tank where the pressed juice is chiller to 6-15° C. and pH adjusted to 4.0 using phosphoric acid.

Aprotinin clarification was performed by two methods prior to ceramic filtration trials. The centrifugation method described in the “Background of Invention” section was performed first with a recovery rate of about 50%. Clarification by means of a rotary drum vacuum filter using diatomaceous earth as a filter aid was also performed on several early production runs. The rotary drum system had recovery rates around 64%. Representative recovery rates for each system are listed below:

TABLE 1
Centrifugation Method

| Centrifuge | Gel |
|---|---|
| Feed | 100% |
| S1 | 49% |

TABLE 2
Diatomaceous Earth Method - Lot 1

| Rotary Drum | | |
|---|---|---|
| Vacuum Filter | | |
| Florida Excelsiana | | |
| 100 L Wash | Gel | Trypsin |
| Feed | 100% | 100% |
| Filtrate | 43% | 51% |
| Filtrate + Wash | 62% | 64% |

TABLE 3
Diatomaceous Earth Method - Lot 2

| Rotary Drum | | |
|---|---|---|
| Vacuum Filter | | |
| Greenhouse | | |
| Excelsiana | | |
| 100 L Wash | Gel | Trypsin |
| Feed | 100% | 100% |
| Filtrate | 24% | 40% |
| Filtrate + Wash | 36% | 53% |

Initial ceramic filtrations of green juice homogenate obtained as described above were conducted utilizing a pilot scale skid from Pall Inc. (East Hills, N.Y.). This system was configured in a closed loop batch mode, with two ceramic membrane holders in series. The first ceramic module contained a Pall 0.2 micron 1P 19-40 element and the second module contained a Pall 0.1 micron 1P 19-40 element. The skid was equipped with a heat exchanger. Specific characteristics and operating parameters of the pilot scale system are as follows:

TABLE 4
Pall Pilot Skid Characteristics/Operating Parameters

| | | |
|---|---|---|
| Element area | .24 sq. m each, total .48 Sq. m | |
| Pore size | Module 1 (.2 micron) | Module 2 (.1 micron) |
| Pump type | Diaphragm pump | |
| Inlet pressures | Module 1 | Module 2 |
| | (35 psi) | (19 psi) |
| Outlet | Module 1 | Module 2 |
| pressures | (19 psi) | (13 psi) |
| Flow rate | 12-15 gpm | |
| Average flux | 51 lm²h | |
| Operating temp | Maintained below 60 F. | |
| | throughout process | |

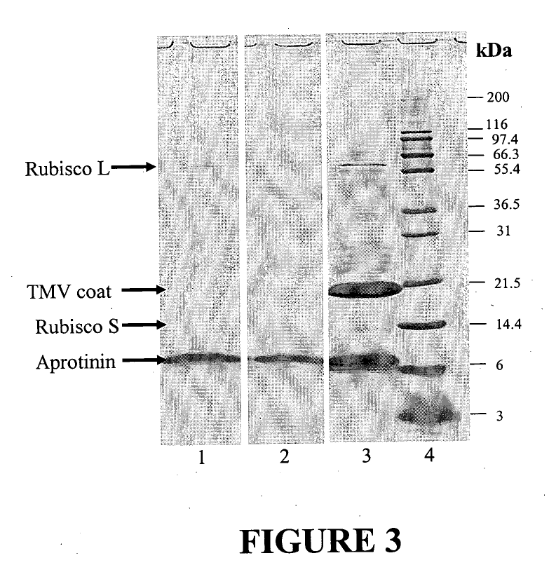

The pilot scale process produced a clear to amber permeate that was filtered through a 5 micron capsule filter prior to ultrafiltration. The permeate sample was analyzed by SDS-PAGE gel. The ceramic filters of 0.2 micron and 0.1 micron efficiently separated aprotinin from the TMV and RuBisCO, as shown in FIG. 3. In particular, FIG. 3 shows permeate sampled after the homogenate was passed through the 0.2 micron filter, and permeate sampled after the homogenate was passed through the 0.1 micron filter.

Samples were also assayed for aprotinin activity using the trypsin inhibition assay described in Fritz, H., Hartwich, G., Werle, E., (1966) Hoppe-Seylers Zeitschrift Für Physiologishche Chemie (Berlin) 345, 150-167 Kassell, B. (1970) Methods in Enzymology XIX, 844-852. Results for two different-lots of plant material are shown below.

TABLE 5
Recovery Rates for Filtration on Ceramic Pilot Skid - Lot 1

| Florida Excelsiana | | |
|---|---|---|
| 60 L Wash | Gel | Trypsin |
| Feed | 100% | 100% |
| Permeate | 69% | 68% |
| Concentrate | 21% | 23% |
| Permeate + Wash | 73% | 74% |

TABLE 6
Recovery Rates for Filtration on Ceramic Pilot Skid - Lot 2

| Florida Excelsiana | | |
|---|---|---|
| 21 L Wash | Gel | Trypsin |
| Feed | 100% | 100% |
| Permeate | 62% | 72% |
| Concentrate | 11% | 18% |
| Permeate + Wash | 72% | 83% |

The results of pilot scale aprotinin purification procedures, as described above, with two other lots of plant tissue grown in the greenhouse are shown below. FIG. 1 shows a SDS-PAGE gel of samples from the aprotinin purification summarized in Table 8.

TABLE 7
Aprotinin processing recovery from N. Excelsiana tissue using
ceramic filtration technique by trypsin inhibition assay
Process Yield

| | | mg/ | | |
|---|---|---|---|---|
| Sample | Volume Liter | mL | total g | Recovered |
| GJ (total) | 160 | 0.23 | 37.4 | |
| Ceramic Permeate | 298 | 0.09 | 26.5 | 71% |
| Ceramic Retentate | 62 | 0.11 | 7.0 | 19% |
| UF DF Pool | 20.3 | 1.32 | 26.8 | 72% |

TABLE 8
Aprotinin processing recovery from N. bethamiana tissue using
ceramic filtration technique by trypsin inhibition assay.
Step Process Yield

| | | mg/ | | |
|---|---|---|---|---|
| Sample | Volume Liter | mL | total g | Recovered |
| GJ (total) | 105 | 0.31 | 32.8 | |
| Ceramic Permeate | 250 | 0.10 | 24.5 | 75% |
| Ceramic Retentate | 55 | 0.12 | 6.6 | 20% |
| UF DF Pool | 17.5 | 1.28 | 22.4 | 68% |

After demonstrating that 0.1-micron ceramic filtration would be efficient and effective in the aprotinin clarification process a larger skid equipped with two 7P19 0.1 -micron ceramic modules (also referred to herein as filters) was designed. These modules consist of 7 ceramic elements (also referred to herein as membranes) each; the individual elements are identical to the single 0.1 micron element that was used on the pilot skid described above.

This skid is configured to operate in a gravity feed and bleed mode. Feed is pumped from a 1200 liter feed tank through both ceramics modules with permeate (which is the product in this process) flowing into a permeate catch tank and the retentate recycling through the heat exchanger and flowing back into the feed tank. A small percentage of the retentate bleeds directly back to the pump suction to help facilitate higher concentrations without losing pump suction. Specific characteristics and operating parameters of the ceramic filtering skid are as follows:

TABLE 9
LSBC Ceramic Filtering Skid Characteristics/Operating Parameters

| | Module 1 | Module 2 |
|---|---|---|
| Element area | 1.68 sq. m | 1.68 sq. m |
| Pore size | .1 micron | .1 micron |
| Inlet pressures | Module 1 | Module 2 |
| | (45 psi) | (22 psi) |
| Outlet pressures | Module 1 | Module 2 |
| | (22 psi) | (6 psi) |
| Permeate pressures | 15 psi | 4 psi |
| Flow rate | 380-400 lpm | |
| Average flux | 55 lm²h | |
| Operating temp | Maintained below 60 F. | |
| Pump type | Centrifugal | |

Before first use of the skid the ceramic modules were removed for passivation. After passivation and cleaning in place, initial clean water permeability readings were established. Clean water permeability is the standard to determine effectiveness of cleaning.

Determining the minimum hold up volume of the system was carried out by adding a known volume of water and operating the system until the pump lost suction. Permeate volume was then subtracted from the starting feed volume; the difference was the minimum hold up volume. 60 L is the volume that has been determined to be the standard hold up volume.

Through testing and development the most efficient operation occurred at the pressures in the table above. The system was designed to operate at an average liters per square meter per hour (lm²h) of 40. During trials and normal production the skid has averaged over 55 lm²h, which results in an average processing rate of 185 liters per hour of feed material.

After initial production runs using homogenate of plant material infected with the aprotinin virion construct, as described above, there was an indication that recovery percentages were lower than had been expected. Even though a wash volume equal to 2 X concentrate volume was being performed at the end of each run, a significant amount of product was remaining in the concentrate. This was attributed to a combination of typical low volumes of ceramic feed and the 60 L hold up volume. It is optimal to have enough feed material to achieve a 16-20X concentration. Several experiments were conducted with various volumes of wash and samples were taken of each wash separately to determine optimum volumes of wash to maximize recovery. Analysis indicated that 320 L was the most effective volume of wash when comparing recovery percentage gained to increased processing time not only at the ceramic skid but also at the UF process. Each 50 L of wash adds 20 minutes of processing time to the ceramic process and 21 minutes to the UF process. Specific wash experiment data showing percentage recovery aprotinin are listed below in Table 10:

TABLE 10
Wash Experiment Data

| Ceramic Filter Wash | |
|---|---|
| Experiment | Recovery % |
| Feed | 100% |
| Permeate + 120 L Wash | 75% |
| 1st Wash (50 L) | 82% |
| 2nd Wash (50 L) | 73% |
| 3rd Wash (50 L) | 91% |
| 4th Wash (50 L) | 94% |
| 4th Wash (50 L) | 96% |

Through multiple processing runs the ceramic has proven to be consistent from lot to lot. Below is a representative summary of a production run:

TABLE 11
Typical Ceramic Summary

| Lot #05C0015 | % Recovery |
|---|---|
| Feed | 100% |
| Concentrate | 13% |
| Composite | 88% |
| Permeate/Wash | |

### Example 2: Pilot Scale Manufacturing of rAprotinin

Nicotiana excelsiana plants infected with the TMV vector described in Example 1 that encodes aprotinin were harvested, homogenized and pH adjusted as described above in Example 1.

pH adjusted liquid was then clarified by micro-filtration using a skid equipped with two (2) Pall 7-P19-40, 0.1 micron ceramic membrane modules (also referred to as filters), as described above in Table 8. Liquid was processed through this skid until the feed volume/retentate reaches the system minimum hold up volume (approximately 60 L).

A batch wash of the system was then conducted with 320 L of buffer (sodium chloride, ascorbic acid, sodium metabisulfite) to recover additional rAprotinin remaining in the ceramic retentate. The rAprotinin was recovered in the ceramic permeate.

The ceramic permeate was filtered through a 5 micron capsule filter prior to ultra-filtration (UF). Ultra-filtration was accomplished by means of a SETEC ultra-filtration skid equipped with 17 square meters of Millipore 3Kd regenerated cellulose membrane. Product was filtered and concentrated to a minimum10 X concentration factor and then diafiltered with buffer (20 mM sodium phosphate, pH 4.0) until the conductivity reached a level<3 ms. UF retentate was then pumped to a tank chilled at 6-15° C. A wash of the UF system was then conducted using the diafiltration buffer described above to recover any residual rAprotinin remaining in the UF system. The UF wash was then pumped into the tank containing the UF retentate. The 3 Kd UF retentate was then pH adjusted to 6.5 using ION NaOH. The pH adjusted retentate was then filtered through a the 0.2 μm capsule filter.

rAprotinin was further purified by loading the 0.2 μm-filtered 3kD UF-retentate from the extraction process onto a column of SP Sepharose Fast Flow (GE Healthcare) at a ratio of 20 mg rAprotinin/mL of resin. The column was equilibrated in 20 mM sodium phosphate, pH 6.5, and washed to UV baseline with the same buffer after the load is applied. Two elution step gradients were then created by blending 20 mM sodium phosphate, pH 6.5 and 20 mM sodium phosphate, 205 mM NaCl, pH 6.5. The first step gradient generates a NaCl concentration of 130 mM and was used to wash the column to baseline. The second step gradient generates a NaCl concentration of 180 mM and was again used to wash the column to UV baseline. A final elution was then performed using 205mM NaCl to wash the column to UV baseline, the resulting UV peak was collected after filtration through an in-line 0.2 μm capsule filter (Sartorius).

The resulting SP Sepharose pool of rAprotinin was then adjusted to pH 2.7 using 6N HCl and filtered through a 0.2 μm capsule filter (Sartorius). The rAprotinin was then loaded onto a column of Source 15 RPC resin (GE Healthcare) at a ratio of 5 mg rAprotinin/mL of RPC resin. The column was equilibrated in 25 mM potassium phosphate, pH 2.7. After loading, the column was washed with 1 Column Volume (CV) of 25 mM potassium phosphate, pH 2.7, 1.5% n-propanol. The column was washed with a linear gradient from 1.5% to 4.1% n-propanol over 3 CV. The wash was then held at 4.1% n-propanol for 6 CV. Following this hold step, the column was washed with a linear gradient from 4.1% to 5.6% n-propanol over 8 CV. The wash was subsequently held at an n-propanol concentration of 5.6% for an additional 15 CV. Following this hold step, the rAprotinin was eluted from the column using a linear gradient from 5.6% to 12.0% n-propanol over 12 CV. Following the elution, the column was washed with 5 CV of 15% n-propanol buffer to ensure that all desired rAprotinin was recovered from the column. Finally, the column was stripped of protein by washing with 5CV of a 65% n-propanol solution. All collected samples were tested for % purity, and % oxidation. Samples exhibiting greater than 99% purity and low oxidation were pooled, filtered through a 0.2 μm capsule filter (Sartorius), and stored at 4° C. until the next step of the process.

The resulting Source 15 RPC Pool was loaded into a Sartorius Slice Labtop 200/250 ultra-filtration system. The solution was concentrated to a minimal volume using Sartorius 1K molecular weight cut-off membranes with a total surface area of 0.5 square meters. The retentate was recirculated through the membranes while the permeate was collected in a separate vessel. The retentate and permeates were tested for total protein by A280 absorbance readings. The retentate was diafiltered versus 10 volumes of sterile normal saline. Following the diafiltration step, the pH and conductivity of the retentate should match those of the sterile saline. The diafiltered pool was drained from the system and was filtered through a 0.2 μm capsule filter (Sartorius) into a sterile media bag for further dispensing.

### Example 3: Pilot Scale Purification of Antiviral Protein from Plant Material

In another experiment, a 13 kDa antiviral protein, griffithsin, was purified from plant material. Griffithsin was expressed in Nicotiana benthamiana plants infected with a viral vector derived from tobacco mosaic virus engineered to encode griffithsin. A green juice extract was prepared from N. benthamiana leaves using 30 mM sodium acetate, pH 5, 375 mM NaCl, 0.15% Na meta bisulfite, and 22.5 mM ascorbic acid. Tissue was homogenized at a buffer to biomass ratio of 1L: 2 kg using a disintegrator and then processed through a press to remove cell debris.

The green juice feed with pH 6 was applied to the ceramic filtration skid and operated at 56.3 lm² h average speed. To increase product recovery, the ceramic concentrate was washed with additional extraction buffer, which was passed through the ceramic membrane again, and added to the ceramic permeate initially collected. The ceramic permeate was amber clear and particle free. TMV and RuBisCO were efficiently removed from the extract as revealed by SDS-PAGE analysis shown in FIG. 2.

### Example 4: Concentration of Tobacco Mosaic Virus

A solution of purified wild-type U1 tobacco mosaic virus (TMV) was prepared that contained approximately 1.1. mg/mL of virus in 10 mM sodium-poassium phosphate buffer, pH 7.2. Starting ceramic membrane feed consisted of 1500 mL of this virus solution. A total of about 1000 mL of permeate was collected in 250 mL fractions and passed through a lab-scale ceramic membrane unit. (Pall Life Sciences, X-Lab 3W). This membrane unit uses a 50 square centimeter (0.005 sq. meter) single lumen ceramic membrane coupled with a Jabsco pump and a 3-liter jacketed reservoir. It utilizes a closed circulation loop that is pressurized from a compressed air source to provide the necessary trans-membrane pressure. A back-pulsing device is installed on the permeate outlet which briefly back pressures the membrane to dislodge any accumulated gel layer or solids. The X-Lab unit is self-contained and all necessary gauges and piping are included.

Flux rate started at about 29 mL/min and decreased to 22.5 mL/min throughout the run. The retentate was not foamy and its volume was 475 mL. Samples were assayed for total protein by the BCA assay and by SDS-PAGE using TMV as standards in both cases.

TABLE 12
Total Protein Assays

| Sample | BCA protein mg/mL | SDS-PAGE virus mg/mL |
|---|---|---|
| Ceramic feed | 0.91 | 1.01 |
| Ceramic permeate | 0 | 0.03 |
| Ceramic retentate | 2.86 | 2.68 |

The ceramic permeate was assayed for virus concentration using the Glurk assay. The Glurk assay is described in Holmes, F.O. (1938) Phytopathology, 28, 553-561 and Takahashi, W. N. (1956); Phytopathology 46, 654-656 Glurk results indicated that the concentration of TMV in the permeate is approximately 0.0002 mg/mL.

Deposit Information

The following plasmid was deposited under the terms of the Budapest Treaty with the American Type Culture Collection, 10801 University Blvd., Manassas, Va. 20110-2209, USA (ATCC): Plasmid pLSB2602 is Patent Deposit PTA-6577, deposited Feb. 10, 2005. 100871 This deposit was made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit or 5 years after the last request, whichever is later. The assignee of the present application has agreed that if a culture of the materials on deposit should be found nonviable or be lost or destroyed, the materials will be promptly replaced on notification with another of the same. Availability of the deposited material is not to be construed as a license to practice the invention in contravention of the rights granted under the authority of any government in accordance with its patent laws, or as a license to use the deposited material for research.

What is claimed is:

1. A method for isolating a target compound from a plant, the method comprising:
(a) homogenizing tissue of the plant to produce a green juice homogenate;
(b) passing the green juice homogenate from step (a) through a ceramic filter; and
(c) purifying the target compound from a permeate created in step (b).

2. The method of claim 1, wherein the ceramic filter has a pore size of equal to or less than 5 microns.

3. The method of claim 2, wherein the pore size is less than or equal to 1 micron.

4. The method of claim 3, wherein the pore size is less than or equal to 0.2 micron.

5. The method of claim 4, wherein the pore size is less than 0.1 micron.

6. The method of claim 1, wherein step (a) further comprises adjusting the pH and/or ionic content of the green juice homogenate such that the target compound is soluble.

7. The method of claim 6, wherein the pH is adjusted.

8. The method of claim 6, wherein the ionic strength is adjusted.

9. The method of claim 6, wherein the pH is neutral to acidic.

10. The method of claim 9, wherein the pH is adjusted to equal to or less than about 7.

11. The method of claim 10, wherein the pH is adjusted to equal to or less than about 5.2.

12. The method of claim 1, wherein step (a) further comprises heating the green juice homogenate to between about 45° C. to 65° C.

13. The method of claim 1, wherein step (c) comprises ultrafiltration.

14. The method of claim 1, wherein step (c) comprises chromatography, an affinity-based method of purification, salt precipitation, polyethylene glycol precipitation or crystallization.

15. The method of claim 1, wherein step (c) comprises subjecting the permeate to cation exchange chromatography.

16. The method of claim 15, wherein the cation exchange chromatography comprises a Sepharose SP column.

17. The method of claim 15, wherein step (c) further comprises subjecting an eluant from cation exchange chromatography to reversed phase chromatography.

18. The method of claim 16, wherein the reversed phase chromatography comprises a column selected from the group consisting of RPC 15 and RPC 30.

19. The method of claim 1, wherein step (b) further comprises washing one or more times a concentrate created by passing the green juice homogenate through the ceramic filter.

20. The method of claim 1, wherein the plant is infected with a viral vector.

21. The method of claim 20, wherein the viral vector is derived from tobacco mosaic virus.

22. The method of claim 20 further comprising purifying the viral vector or a portion thereof from a concentrate produced in step (b).

23. The method of claim 1, wherein the target protein is expressed in the plant by a transgene.

24. The method of claim 1, wherein the target compound is a protein.

25. The method of claim 24, wherein the protein is aprotinin.

26. The method of claim 24, wherein the protein is an antiviral protein.

27. The method of claim 24, wherein the target protein is soluble.

28. The method of claim 1, wherein the target compound is selected from the group consisting of sugars, vitamins, alkaloids, flavors, and amino acids.

29. The method of claim 1, wherein in step (b) the ceramic filter comprises more than one ceramic filter arranged in a series.

30. The method of claim 29, wherein the more than one ceramic filter comprises two ceramic filters having pore sizes of 0.1 micron and 0.2 micron.

31. A method for isolating a target protein from a plant, the method comprising:
(a) obtaining a plant extract;
(b) passing the plant extract from step (a) through a ceramic filter; and
(c) purifying the target compound from a permeate created in step (b).

32. The method of claim 31, wherein the plant extract is an interstitial fluid extract.

33. The method of claim 31, wherein the plant extract is a green juice homogenate.

34. A method for purifying virus from a plant extract comprising passing the plant extract through a ceramic filter with a pore size of at least 0.5 micron.

35. The method of claim 34, wherein the pore size is at least 0.2 micron.

36. The method of claim 35, wherein the pore size is at least 0.1 micron.

37. The method of claim 36, wherein the virus is tobacco mosaic virus.
